(19) United States
(12) Patent Application Publication
(10) Pub. No.: US 2026/0234529 A1
(43) Pub. Date: Aug. 13, 2026

(54) CELL CULTURE APPARATUS AND METHODS OF USING SAME

(71) Applicant: STEMCELL TECHNOLOGIES CANADA INC., Vancouver (CA)

(72) Inventors: Michael HIATT, Vancouver (CA); Eric JERVIS, Vancouver (CA); Selena CEN, Vancouver (CA); Douglas KONDRO, Vancouver (CA); Santiago CAMPUZANO, Vancouver (CA)

(21) Appl. No.: 19/469,720

(22) PCT Filed: Apr. 5, 2024
(86) PCT No.: PCT/CA2024/050442

§ 371 Date: Sep. 26, 2025

Related U.S. Application Data
(60) Provisional application No. 63/457,583, filed on Apr. 6, 2023.

(51) Int. Cl. C12M 1/12; C12M 1/32; C12M 3/00

(52) U.S. Cl. CPC C12M 25/14; C12M 21/08; C12M 23/12

(57) ABSTRACT

This disclosure describes cell culture devices having non-standard wall architecture and/or geometries. Cell culture devices as described in this disclosure may include terraced or tiered wall segments, where successive terraces or tiers are dimensionally wider. This disclosure also relates to uses of the described cell culture devices in methods or processes.

### CROSS REFERENCE TO RELATED APPLICATION

This application claims the benefit of United States Provisional Patent Application No. 63/457,583 filed Apr. 6, 2023, the entire content of which is hereby incorporated by reference in its entirety.

### TECHNICAL FIELD

This disclosure relates to cell culture, and more specifically to apparatus for cell culture, and still more specifically to applications or methods using such apparatus for cell culture.

### BACKGROUND

In vitro or ex vivo cell culture systems increasingly rely on culturing cells either within or on an extracellular matrix, because extracellular matrices provide support and/or signals that mimic the in vivo environment. In particular, current organoid and other 3D models tend to rely on support from an extracellular matrix or mimetic.

Organoids and other three dimensional models are becoming widespread in cell culture workflows, perhaps because they represent a more physiologically relevant model system in terms of tissue/organ architecture and cellular composition, in comparison to 2D adherent and non-adherent culture. Indeed, organoids and other three dimensional model systems may enable more accurate responses to drugs and other compounds, simplifying the path to the clinic Further, as a more physiologically relevant model system, organoids and other three dimensional models may be more appropriate for cell therapy and regenerative medicine applications.

While improvements in physiological relevance of three dimensional model systems may arise because they better support higher biological complexity, such three-dimensional systems are often accompanied by increased heterogeneity. On the one hand, heterogeneity may arise for reasons inherent to biological systems, which may of itself be the reason why organoid systems better recapitulate physiological relevance in relation to more traditional systems that support a less diverse range of cell types or lack appropriate spatial organization of cell types. On the other hand, three dimensional approaches are typically more technically challenging and this may give rise to experimental heterogeneity.

The current state of the art of organoid or three-dimensional tissue generation is to form a dome of extracellular matrix comprising cells on the surface of a conventional flat-bottomed culture dish or plate. Such dome culture suffers numerous drawbacks, such as variable mass transfer rates depending on the localization of cells within the dome, and lack of control over dome shape and position on the surface.

Therefore, there is a need for improved cell culture apparatus that enable the biological complexity of three-dimensional culture models while reducing associated technical shortcomings/challenges.

### SUMMARY

This disclosure relates to cell culture, and more specifically to apparatus for cell culture and to applications or methods using such apparatus for cell culture. In certain aspects are described apparatus for culturing cells. In certain aspects are described cell culture methods or processes using apparatus of this disclosure.

In one aspect of this disclosure are provided cell culture apparatus. Cell culture apparatus of this disclosure may comprise a well having a bottom wall and one or more sidewalls to define an opening that is opposed to the bottom wall. In one embodiment, a cell culture apparatus comprises a plurality of sidewall segments having a continuous perimeter.

In one embodiment, a cell culture apparatus (and more particularly a well thereof) comprises a first sidewall segment connected to the bottom wall and extending in a direction away from the bottom wall. The first sidewall segment and the bottom wall bound a first volume, which may hold or retain a liquid and/or a polymerizable liquid.

A cell culture apparatus (and more particularly a well thereof) may further comprise a second sidewall segment connected to the first sidewall segment by a first ledge. The second sidewall segment bounds a space above the first volume such that the second sidewall segment and a plane of the first ledge bounds a second volume, which may hold or retain a liquid and/or a polymerizable liquid (provided that the first volume is filled with a liquid or a polymerizable liquid).

In one embodiment, a height of the first sidewall segment is greater than a height of the second sidewall segment.

In one embodiment, the first volume is equal +10% to the second volume. In one embodiment, the first volume and the second volume are substantially equal or equal.

In one embodiment, the height of the first sidewall segment is between about 0.1 mm and 10 mm, preferably between 0.5 mm and 5 mm. In the same or different embodiment, the height of the second sidewall segment is between about 0.1 mm and 10 mm, preferably between 0.2 mm and 4 mm. In one embodiment, a height of the first sidewall segment is greater than a height of the second sidewall segment.

In one embodiment, the first sidewall segment forms an obtuse angle with the bottom wall, and/or the second sidewall segment forms an obtuse angle with the first ledge. In one embodiment, the angle is between 90 degrees and 100 degrees.

In one embodiment, a width of the first ledge is between about 0.1 mm to about 5 mm. In one embodiment, a width of the first ledge is about 1-2 mm±0.25 mm.

In one embodiment, one or both of the first sidewall segment and the second sidewall segment are substantially circular in the plane of the bottom wall (e.g. when viewed in a direction of the opening toward the bottom wall). In one embodiment, a diameter of the first sidewall segment at any position along the height thereof is smaller than a diameter of the second sidewall segment at any position along the height thereof. The diameter of the first and second sidewall segments depends on the plate format.

In one embodiment, a diameter of the first sidewall segment (along the widest plane) is between about 2 mm and 35 mm. In one embodiment, a diameter of the first sidewall segment (along the widest plane) is between about 2 mm and 30 mm. In one embodiment, a diameter of the first sidewall segment (along the widest plane) is between about 2 mm and 25 mm. In one embodiment, a diameter of the first sidewall segment (along the widest plane) is between about 2 mm and 20 mm. In one embodiment, a diameter of the first sidewall segment (along the widest plane) is between about 2 mm and 15 mm. In one embodiment, a diameter of the first sidewall segment (along the widest plane) is between about 2 mm and 10 mm.

In one embodiment, a diameter of the second sidewall segment (along the widest plane) is between about 1 mm and 40 mm. In one embodiment, a diameter of the second sidewall segment (along the widest plane) is between about 2 mm and 35 mm. In one embodiment, a diameter of the second sidewall segment (along the widest plane) is between about 2 mm and 30 mm. In one embodiment, a diameter of the second sidewall segment (along the widest plane) is between about 2 mm and 25 mm. In one embodiment, a diameter of the second sidewall segment (along the widest plane) is between about 2 mm and 20 mm. In one embodiment, a diameter of the second sidewall segment (along the widest plane) is between about 2 mm and 15 mm. In one embodiment, a diameter of the second sidewall segment (along the widest plane) is between about 2 mm and 10 mm. In one embodiment, the diameter of the second sidewall segment is between about 3 mm and 15 mm.

Cell culture apparatus, and particularly a well thereof, may further comprise a third sidewall segment connected to the second sidewall segment by a second ledge. The third sidewall segment a bounds a space above the second volume such that the third sidewall segment and a plane of the second ledge bounds a third volume, which may hold or retain a liquid and/or a polymerizable liquid (provided that the first and second volumes are filled with a liquid or a polymerizable liquid).

In one embodiment, the third sidewall portion extends from the second ledge toward the opening. In one embodiment, the third sidewall portion extends from the second ledge to the opening.

In one embodiment, a height of the third sidewall segment is greater than the height of the second sidewall segment, and the third volume is greater than the second volume. In one embodiment, the height of the third sidewall segment is between about 1 to 20 mm (±1 mm), between about 1 to 15 mm (+1 mm), or between about 1 to 10 mm (±1 mm).

In one embodiment, the second ledge and the third sidewall segment form an obtuse angle. In one embodiment, the angle is between 90 degrees and 100 degrees.

In one embodiment, a width of the second ledge is between about 0.2 mm and about 5 mm.

In one embodiment, the third sidewall segment is circular or substantially circular in the plane of the bottom wall (e.g. when viewed in a direction of the opening toward the bottom wall). In one embodiment, a diameter of the third sidewall segment at any position along the height thereof is larger than the diameter of the second sidewall segment at any position along the height thereof.

Cell culture apparatus of this disclosure may further comprise more than one well. In one embodiment, each well is substantially as described by the foregoing.

Thus, in another aspect of this disclosure are provided cell culture apparatus, comprising a plurality of wells having a bottom wall and an opening. In certain embodiments, more than one of the plurality of wells, or all of the plurality of wells, may comprise a first sidewall segment connected to the bottom wall and extending in a direction away from the bottom wall. The first sidewall segment and the bottom wall bounding a first volume for holding a liquid and/or a polymerizable liquid.

Cell culture apparatus of this aspect may further comprise a second sidewall segment connected to the first sidewall segment by a first ledge. The second sidewall segment bounds a space above the first volume such that the second sidewall segment and a plane of the first ledge bounds a second volume, which may hold or retain a liquid and/or a polymerizable liquid (provided that the first volume is filled with a liquid or a polymerizable liquid).

In one embodiment, a height of the first sidewall segment is greater than a height of the second sidewall segment.

In one embodiment, the first volume is equal or substantially equal to the second volume.

In another aspect of this disclosure are provided methods of culturing cells comprising seeding a population of cells in a well of a cell culture apparatus as described herein.

Methods of this disclosure may further comprise adding a liquid extracellular matrix, one or more liquid extracellular matrix proteins, or a liquid hydrogel to the first volume bounded by the first sidewall segment of the cell culture apparatus. In one embodiment, adding the liquid extracellular matrix, one or more liquid extracellular matrix proteins, or a liquid hydrogel comprises filling the first volume bounded by the first sidewall segment with the liquid extracellular matrix, the one or more liquid extracellular matrix proteins, or the liquid hydrogel.

Methods of this disclosure may further comprise polymerizing the liquid extracellular matrix, the one or more liquid extracellular matrix proteins, or the liquid hydrogel, or allowing the liquid extracellular matrix, the one or more liquid extracellular matrix proteins, or the liquid hydrogel to polymerize.

In one embodiment, the population of cells are suspended and/or embedded in the extracellular matrix, the one or more extracellular matrix proteins, or the hydrogel.

Methods of this disclosure may further comprise exposing or contacting the population of cells to or with a cell culture medium by adding the cell culture medium to the second volume bounded by the second sidewall segment. In one embodiment, the second volume bounded by the second sidewall segment is filled with the cell culture medium.

In one embodiment, a volume of the extracellular matrix, the one or more extracellular matrix proteins, or the hydrogel is equal or substantially equal to a volume of the cell culture medium.

Methods of this disclosure may further comprise forming or generating a multicellular aggregate or an organoid from the population of cells.

Methods of this disclosure may further comprise assaying the population of cells. In one embodiment, the assay may be an automated assay such as via an automated liquid handler and/or an automated imager. The types of assays to be performed using the apparatus or by practicing the methods are not particularly limited, and include cell toxicity assays, compound screening assays, growth condition optimization assays, differentiation assays, etc.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows an embodiment of a cell culture apparatus of this disclosure in perspective, cross-sectional view (A) and in a top view with a culture of cells in the first volume (B). Darker shading in (A) may correspond to extracellular matrix or an extracellular matrix component, and incrementally lighter shading thereabove may correspond to cell culture medium or a second layer of extracellular matrix or extracellular matrix component.

FIG. 2 shows enlarged views of an embodiment of a well comprised in a cell culture apparatus of this disclosure in top plan view (A) and in isolated cross-sectional view taken through line A-A (B).

FIG. 3 shows enlarged views of a different embodiment of a well comprised in a cell culture apparatus of this disclosure in top plan view (A) and in isolated cross-sectional view taken through line A-A (B).

FIG. 4 shows enlarged views of a still different embodiment of a well comprised in a cell culture apparatus of this disclosure in top plan view (A) and in isolated cross-sectional view taken through line A-A (B).

FIG. 5 shows line graphs of multicellular structures growth in different embodiments of cell culture apparatus of this disclosure. Growth of hepatic (A) and colon (B) human organoids in extracellular matrix was assessed over several days. Height (depth) of first sidewall portion was varied as indicated. Growth of organoids in cell culture apparatus of this disclosure is shown in comparison to growth of organoids in a standard dome culture. Data represent mean±SD.

FIG. 6 shows characteristics of multicellular structures grown in cell culture apparatus of this disclosure. Line graphs showing the growth of colon organoids over time in either a cell culture apparatus of this disclosure (“Improved”) or in a standard dome culture (“Dome”) (A). Data represent mean±SD, n=4, p<0.001 by 2-way ANOVA with Tukey for multiple comparison. Graph of plating efficiency for colon organoids seeded in either a cell culture apparatus of this disclosure or in a standard dome culture after 7 days in culture. Plating efficiency was calculated by dividing the number of clumps seeded at DO by the counted number of organoids greater than 100 μm in diameter after 7 days in culture (B). Data represent mean±SD, n=4, p<0.001 by T-test. Histogram of human intestinal organoid size distribution after 7 days in culture in either a cell culture apparatus of this disclosure or in a standard dome culture (C).

FIG. 7 shows line graphs of organoid responsiveness to forskolin. Percent swelling of normal human intestinal organoids in either a 24-well format cell culture apparatus of this disclosure or a standard dome culture, following ^{~}90 minutes of exposure to 5 μM forskolin (FSK) (A) or a DMSO control (B). Data represent mean±SD, n=4. Percent swelling of normal human intestinal organoids in either a 96-well format cell culture apparatus of this disclosure or a standard dome culture, following ^{~}90 minutes of exposure to different doses of forskolin (FSK) (C). Data represent mean±SD, n=3, p<0.01 by 2-way ANOVA.

FIG. 8 shows representative images of human hepatic organoids formed/differentiated in varying concentrations of extracellular matrix. Organoids were seeded in either 100%, 75%, 50%, 20% or 10% Matrigel mixed with culture medium into either a 24-well plate format of a cell culture apparatus of this disclosure or as dome cultures on a standard 24-well plate, and imaged at day 4 (A) and at day 7 (B). Scale bars are either 1000 μm in (A) or 200 μm in (B).

FIG. 9 shows graphs quantifying levels of evaporation from the wells of 24-well (A) or 96-well (B) cell culture apparatus of this disclosure in comparison to corresponding wells of standard 24 -and 96-well plates. Individual data points represent experimental replicates, and the horizontal dashes represent the mean±SD.

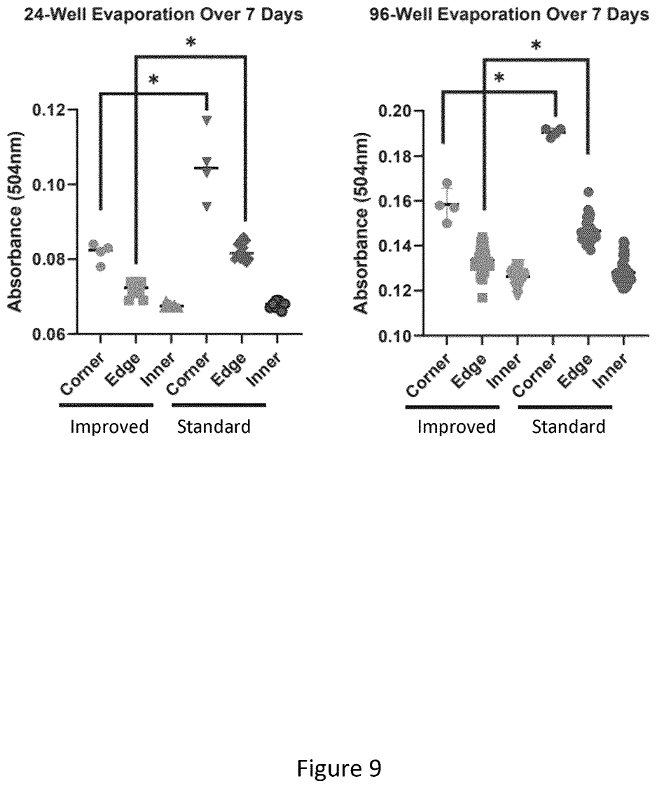

### DETAILED DESCRIPTION

This disclosure relates to cell culture, and more specifically to apparatus for cell culture and to applications or methods using such apparatus for cell culture. In certain aspects are described apparatus for culturing cells. In certain aspects are described cell culture methods or processes using apparatus of this disclosure.

Various apparatus, systems, and methods are described below to provide an example of at least one embodiment of the claimed subject matter. No embodiment described below limits any claimed subject matter and any claimed subject matter may cover apparatus, systems and methods that differ from those described below. The claimed subject matter are not limited to systems, apparatus and methods having all of the features of any one system, apparatus or method described below or to features common to multiple or all of the systems, apparatus and methods described below. Subject matter that may be claimed may reside in any combination or sub-combination of the elements or process steps disclosed in any part of this document including its claims and figures. Accordingly, it will be appreciated by a person skilled in the art that a system, apparatus or method disclosed in accordance with the teachings herein may embody any one or more of the features contained herein and that the features may be used in any particular combination or sub-combination that is physically feasible and realizable for its intended purpose.

Furthermore, it is possible that systems, apparatus or methods described below is not an embodiment of any claimed subject matter. Any subject matter that is disclosed in a system, apparatus or method described herein that is not claimed in this document may be the subject matter of another protective instrument, for example, a continuing patent application, and the applicant(s), inventor(s) and/or owner(s) do not intend to abandon, disclaim, or dedicate to the public any such invention by its disclosure in this document.

It will also be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures, and components have not been described in detail so as not to obscure the example embodiments described herein. Also, the description is not to be considered as limiting the scope of the example embodiments described herein.

It should be noted that terms of degree such as “substantially”, “about” and “approximately” as used herein mean a reasonable amount of deviation of the modified term such that the result is not significantly changed. These terms of degree should be construed as including a deviation of the modified term, such as 1%, 2%, 5%, or 10%, or ±0.1 mm, ±0.5 mm, or ±1 mm, for example, if this deviation would not negate the meaning of the term it modifies.

Furthermore, the recitation of any numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation up to a certain amount of the number to which reference is being made, such as 1%, 2%, 5%, or 10%, or ±0.1 mm, ±0.5 mm, or ±1 mm, for example, if the end result is not significantly changed.

It should also be noted that, as used herein, the wording “and/or” is intended to represent an inclusive—or. That is, “X and/or Y” is intended to mean X or Y or both, for example. As a further example, “X, Y, and/or Z” is intended to mean X or Y or Z or any combination thereof.

Where used in this disclosure, the term “cell culture” or “culturing cells” refers to in vitro or ex vivo expansion, propagation, maintenance, self-organization, differentiation, treatment, and/or assaying of one or more cells in a container or vessel. Cell culture typically involves or occurs in a culture environment comprising a culture medium. A cell culture environment may further comprise an extracellular matrix, one or more extracellular matrix proteins, a (synthetic) hydrogel, or a layer of feeder cells.

Where used in this disclosure, the term “extracellular matrix”, “extracellular matrix protein” or “ECM” refers to molecules that provide structural and/or biochemical support to surrounding cells. An extracellular matrix may comprise an interlocking mesh of fibrous proteins and/or polysaccharides glycosaminoglycans. ECMs are also widely known to promote cell adhesion, cell-to-cell communication and differentiation within a given tissue or agglomerate of cells. Both natural (e.g. produced or secreted by cells) and synthetic (e.g. synthesized from chemical and/or protein components) extracellular matrices are contemplated within the present disclosure. An extracellular matrix may comprise one or more extracellular matrix proteins. Components of an extracellular matrix, and thus an extracellular matrix within the scope of this application, may include one or more of the following: a fibronectin, a laminin, a vitronectin, a tenascin, an entactin, a thrombospondin, an elastin, a gelatin, a collagen, a fibrillin, a merosin, an anchorin, a chondronectin, a link protein, a bone sialoprotein, an osteocalcin, an osteopontin, an epinectin, a hyaluronectin, an undulin, an epiligrin, a kalinin, a synthetic polymer-based hydrogel (such as a polyethylene glycol, a polyvinyl, or any derivative or analogue thereof), a plant-based hydrogel (such as a cellulose, a hemicellulose, a lignin, a starch and a pectin, or any derivative or analogue thereof), a proteoglycan (e.g., heparan sulfate, chondroitin sulfate, keratin sulfate), glycosaminoglycans, or hyaluronic acid. In one embodiment, an extracellular matrix or matrix component (which may be used in or with a culture medium) described herein may be a gelatinous protein and/or polysaccharide mixture secreted by cells, such as fibroblasts, chondrocytes, or Engelbreth-Holm-Swarm (EHS) mouse sarcoma cells. In some embodiments, the extracellular matrix is Matrigel. ECM proteins of this disclosure may be coated on a cell culture surface, such as a bottom wall of a vessel, dish, flask, or plate.

Where used in this disclosure, the term “organoid” refers to a multicellular structure that may be generated in vitro or ex vivo from a starting population of cells, such as primary cells or pluripotent stem cells. Organoids tend to exhibit higher-level organization, reminiscent of the organization observed in a corresponding tissue. Organoids corresponding to various tissue types may be formed using, for example, kits and protocols commercialized by STEMCELL Technologies. Exemplary organoid kits include IntestiCult™, HepatiCult™, STEMdiff™ Intestinal Organoid, or STEMdiff™ Blood vessel Organoid kits. This disclosure primarily focuses on epithelial organoids, but is not necessarily limited to only epithelial organoids. Examples of epithelial tissues that may be formed into the organoids of this disclosure (in accordance with the media and methods disclosed herein) include colon organoids, hepatic organoids, intestinal organoids, pancreatic organoids, or otherwise.

### Apparatus

In one aspect of this disclosure are provided cell culture apparatus, or said another way apparatus for culturing cells.

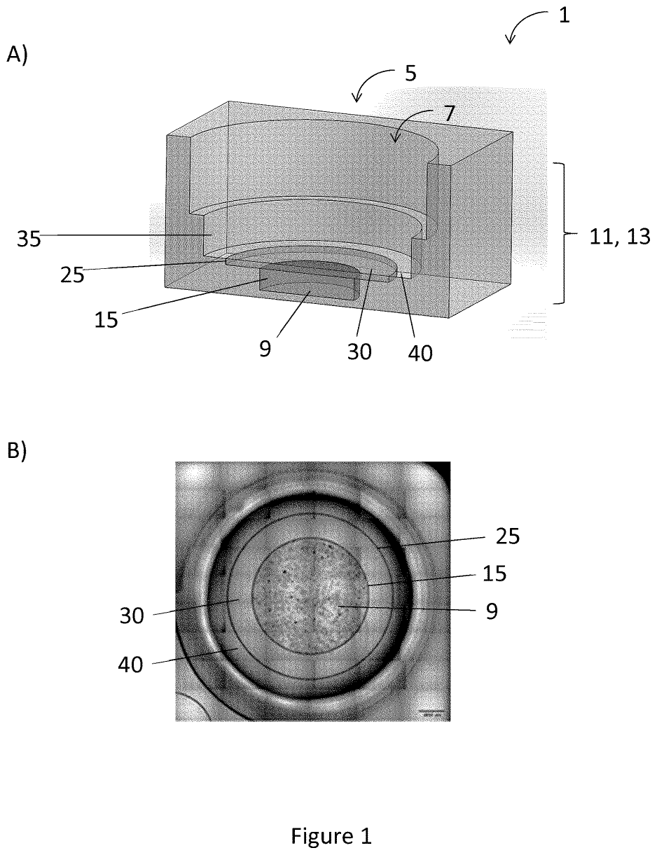

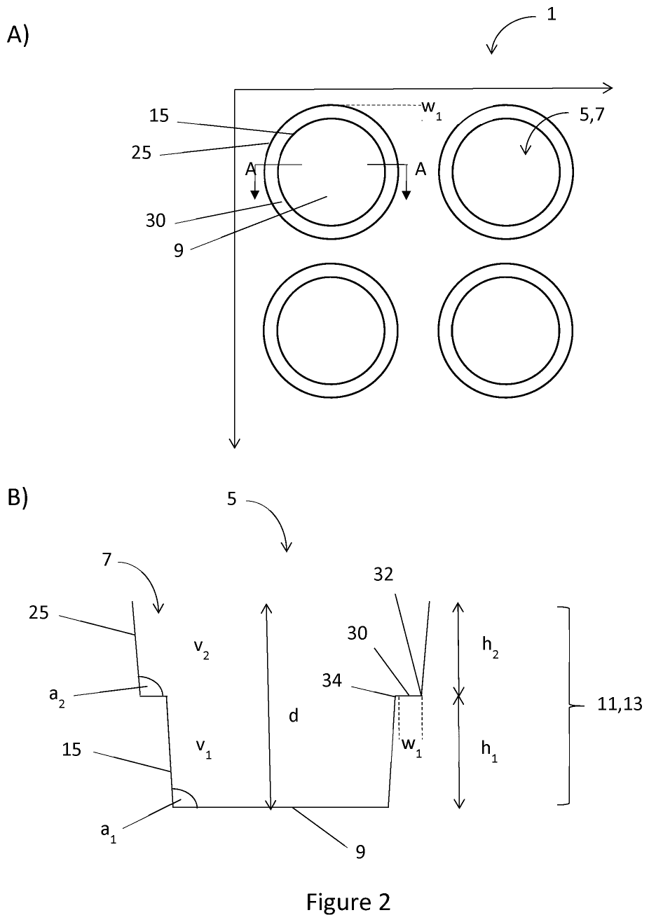

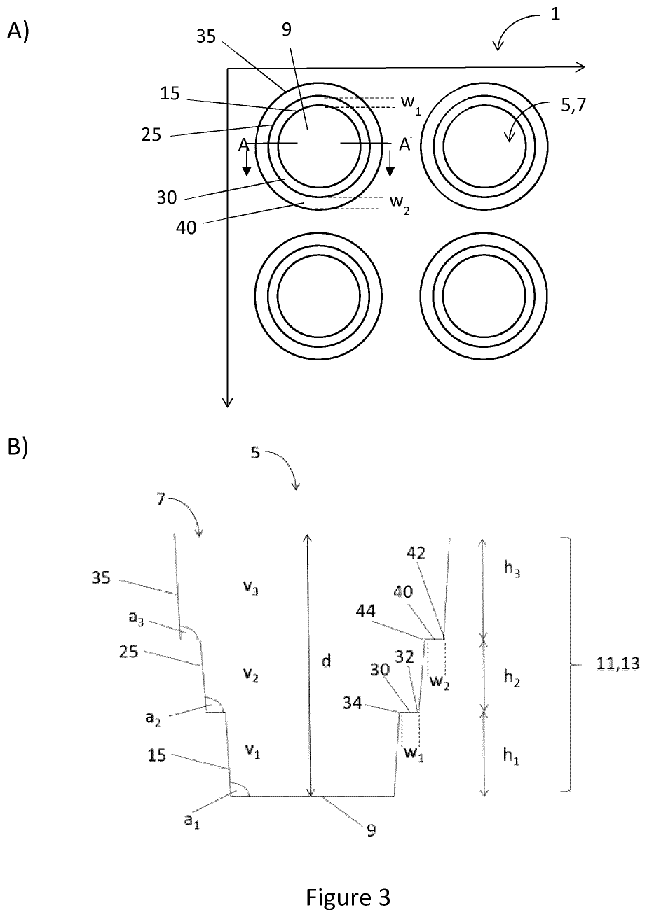

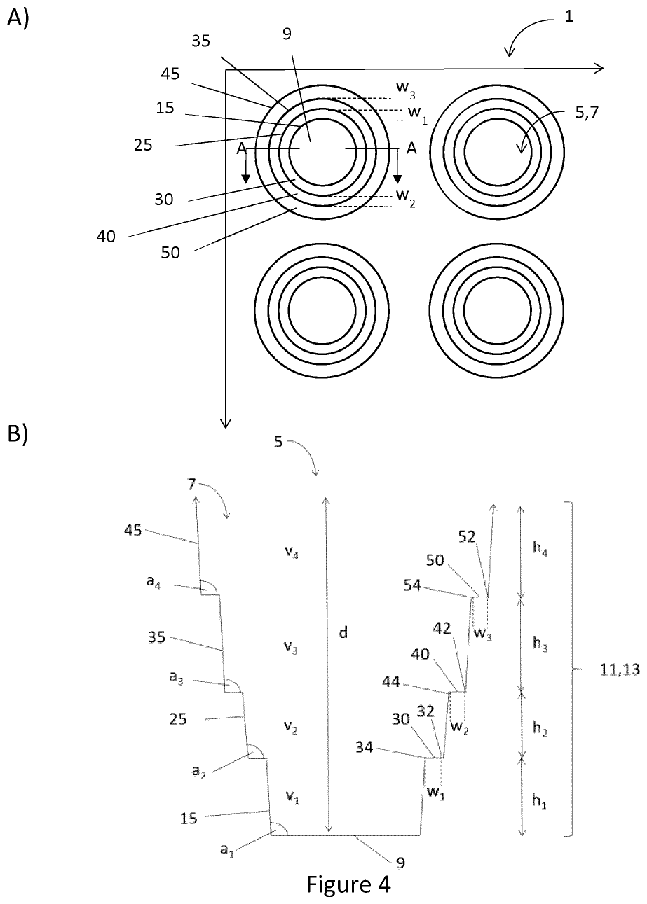

With reference to FIGS. 1-4, cell culture apparatus 1 of this disclosure comprises a well 5 having an opening 7 and a bottom wall 9. Well 5 may further comprise one or more sidewall 11 connecting bottom wall 9 to the (opposed) opening 7. In some embodiments, cell culture apparatus 1 comprises a plurality of such wells 5.

Cell culture apparatus 1 is not limited in terms of the material(s) from which it is constructed or formed, provided that such material(s) are not toxic to a culture of cells. In one embodiment, cell culture apparatus 1 is made of a polymer or a plastic, such as polystyrene, polymethylpentene, acrylic, polypropylene, polycarbonate, or the like. In one embodiment, cell culture apparatus 1 is made of glass or a silicate. In one embodiment, different portions of cell culture apparatus 1 are made of different materials. For example, a base of cell culture apparatus may be made of glass or a silicate, and a well-defining member attached to the base may be made of a polymer or a plastic, or vice versa. In a preferred embodiment, cell culture apparatus is made of a single, integral construction, which may be amenable to injection molding or 3D printing, such as a plastic or a polymer.

Sidewall 11 of cell culture apparatus 1 may be organized into a plurality of sidewall segments or portions 13, each of which cooperate to connect bottom wall 9 to opening 7. Plurality of sidewall segments 13 may comprise a first sidewall segment 15 and a second sidewall segment 25. Each of first sidewall portion 15 and second sidewall portion 25 may be continuous, that is first sidewall portion 15 and second sidewall portion 25 are made of a single material and form a perimeter about well 5.

A cross-sectional shape of first sidewall segment 15 (taken in the plane of bottom wall 9 and/or opening 7) is not particularly limited. In one embodiment, a cross-sectional shape of first sidewall segment 15 is rounded or circular, when viewed in an axis from opening 7 to bottom wall 9 (e.g. in the plane of bottom wall 9 and/or opening 7).

A cross-sectional shape of second sidewall segment 25 (taken in the plane of bottom wall 9 and/or opening 7) is not particularly limited. In certain embodiments, a cross-sectional shape of first sidewall segment 15 is the same as a cross-sectional shape of second sidewall segment 25. In one embodiment, a cross-sectional shape of second sidewall portion 25 is rounded or circular, when viewed in an axis from opening 7 to bottom wall 9 (e. g in the plane of bottom wall 9 and/or opening 7).

In a specific embodiment where first sidewall segment 15 and second sidewall segment 25 are rounded or circular, they are concentric.

If circular, a diameter (at the widest plane or distance across opposed walls) of first sidewall segment 15 may be between about 2 mm and 35 mm, between about 2.25 mm and 30 mm, between about 2. 5 mm and 25 mm, between about 2.75 mm and 20 mm, or between about 3 mm and 15 mm. In one embodiment, a diameter (at the widest plane or distance across opposed walls) of first sidewall segment 15 is between about 2.5 mm and 10 mm (+1 mm), or between about 3 mm and 9 mm (±1 mm).

Also if circular, a diameter (at the widest plane or distance across opposed walls) of second sidewall segment 25 may be between about 1 mm and 40 mm, between about 1.25 mm and 35 mm, between about 1. 5 mm and 30 mm, between about 1.75 mm and 25 mm, or between about 2 mm and 20 mm. In one embodiment, a diameter (at the widest plane or distance across opposed walls) of second sidewall segment 15 is between about 4 mm and 13 mm (±1 mm), or between about 5 mm and 12 mm (±1 mm). In one embodiment, a diameter (at the widest plane or distance across opposed walls) of second sidewall segment 15 is between about 5 mm and 33 mm, or between about 3.5 mm and 13 mm, or between about 4 mm and 12 mm.

In embodiments where well 5 is comprised in a 96-well plate format, a diameter of first sidewall segment 15 (at the widest plane or distance across opposed walls) may be between about 2 mm and about 5 mm (±0.5 mm), between about 2.5 mm and about 4.5 mm (±0.5 mm), or between about 3 mm and about 4 mm (±0.5 mm). In such embodiments, a diameter of second sidewall segment 25 may be between about 3 mm and about 7 mm (±0.5 mm), between about 3.5 mm and about 6 mm (±0.5 mm), or between about 4 mm and about 5 mm (±0.5 mm).

In embodiments where well 5 is comprised in a 24-well plate format, a diameter of first sidewall segment 15 (at the widest plane or distance across opposed walls) may be between about 5 mm and about 12 mm (±0.5 mm), between about 5.5 mm and about 11 mm (±0.5 mm), between about 6 mm and about 10 mm (±0.5 mm), or between about 5.5 mm and about 9.5 mm (±0.5 mm). In such embodiments, a diameter of second sidewall segment 25 may be between about 7.5 mm and about 15 mm (±0.5 mm), between about 8.5 mm and about 14 mm (±0.5 mm), between about 9 mm and about 13.5 mm (±0.5 mm), between about 9.5 mm and about 13 mm (±0.5 mm), or between about 10 mm and about 12.5 mm (±0.5 mm).

In some embodiments well 5 is comprised in a plate format other than a 96- or 24-well, such as a 48-well plate, a 12-well plate, a 6-well plate, or a petri-type dish, and in such cases a diameter of first sidewall portion 15 and of second sidewall portion 25 may be as set out in the foregoing or may scale accordingly.

First sidewall segment 15 is connected to bottom wall 9 and extends therefrom in a direction away from bottom wall 9, such as toward or in the direction of opening 7. Thus, first sidewall segment 15 circumscribes bottom wall 7, and together first sidewall segment 15 and bottom wall 9 bound a first volume v₁ for retaining/holding a liquid and/or a polymerizable liquid, such as a liquid comprising an extracellular matrix or extracellular matrix protein.

A height h₁ of first sidewall segment 15 is only limited by the dimension of cell culture apparatus 1, and more specifically by a depth of well 5. In one embodiment, height h₁ of first sidewall segment 15 is between about 0.5 mm and about 5 mm, between about 0.6 mm and about 4 mm, between about 0.7 mm and about 3 mm, between about 0.8 mm and about 2.5 mm, or between about 0.9 mm and about 2 mm.

In some embodiments, height h₁ of first sidewall segment 15 is the same or substantially the same regardless of whether well 5 is comprised in a 96-well format plate or a 24-well format plate. In some embodiments, height h₁ of first sidewall segment 15 is different when well 5 is comprised in a 96-well format plate or a 24-well format plate. In one embodiment, height h₁ of first sidewall segment 15 is larger when well 5 is comprised in a 24-well format plate compared to when it is comprised in a 96-well format plate.

In some embodiments well 5 is comprised in a plate format other than a 96- or 24-well, such as a 48-well plate, a 12-well plate, a 6-well plate, or a petri-type dish, and in such cases height h₁ of first sidewall segment 15 and of second sidewall segment 25 may be the same as set out in the foregoing or may scale accordingly, or may be different.

Second sidewall segment 25 is situated above first sidewall portion 15 when cell culture apparatus 1 is viewed normal to the depth axis d of well 5. Nevertheless, second sidewall segment 25 is connected to first sidewall segment 15 by a first ledge 30 that extends outward of first sidewall segment 15, relative to a center point of bottom wall 9. At the point where second sidewall segment connects first ledge 30, second sidewall segment 25 extends therefrom toward or in the direction of opening 7.

Thus, in certain embodiments second sidewall segment 25 is dimensionally wider (taken in the plane of bottom wall 9 and/or opening 7) than first sidewall segment 15. Regardless of the shape of the first and second sidewall portions (as viewed in the plane of bottom wall 9 and/or opening 7), if not otherwise stated, for the purposes of this description a measure of distance between two points on a respective sidewall segment is taken where the measure is maximal. Thus, in the case of a quadrilateral the measure is taken along the longest edge, and in the case of a circle the measure is taken along the diameter. Accordingly, if circular a diameter of second sidewall segment 35 is greater than a diameter of first sidewall segment 15.

In one embodiment, a cross-sectional shape of second sidewall segment is polygonal (as viewed in the axis from opening 7 toward bottom wall 9), such as a quadrilateral, pentagon, hexagon, octagon, and so on. In one embodiment, a cross-sectional shape of second sidewall segment is rounded or circular (as viewed in the axis from opening 7 toward bottom wall 9).

Where apparatus 1 comprises a first sidewall segment 15 and a second sidewall segment 25, first ledge 30 connects a bottom end/edge 32 (i.e. the end/edge that is closer to bottom wall 9) of second sidewall segment 25, and an upper end/edge 34 (i.e. the end/edge furthest from bottom wall 9) of first sidewall segment. In one embodiment, a width of first ledge w₁ may be between about 0.1 mm and about 5 mm (±0.5 mm), between about 0.2 mm and about 4 mm (±0.5 mm), between about 0.3 mm and about 3 mm (±0.5 mm), or between about 0.4 mm and about 2 mm (±0.5 mm).

In embodiments where well 5 is comprised in a 96-well plate format, a width of first ledge w₁ may be between about 0.1 mm and about 1 mm (±0.5 mm), between about 0.2 mm and about 0.9 mm (±0.5 mm), between about 0.3 mm and about 0.8 mm (±0.5 mm), or between about 0.4 mm and about 0.7 mm (±0.5 mm).

In embodiments where well 5 is comprised in a 24-well plate format, a width of first ledge w₁ may be between about 0.5 mm and about 5 mm (±0.5 mm), between about 0.6 mm and about 4 mm (±0.5 mm), between about 0.7 mm and about 3 mm (±0.5 mm), or between about 0.8 mm and about 2 mm (±0.5 mm).

In some embodiments well 5 is comprised in a plate format other than a 96- or 24-well, such as a 48-well plate, a 12-well plate, a 6-well plate, or a petri-type dish then width of first ledge w₁ may be as set out in the foregoing or may scale accordingly.

A height h₂ of second sidewall segment 25 is only limited by the dimension of cell culture apparatus 1, and more specifically by a depth of well 5. In some embodiments, a height h₂ of second sidewall segment is dependent on a height h₁ of first sidewall segment 15. In one embodiment, height h₂ of second sidewall segment 25 is between about 0.2 mm and about 2 mm, between about 0.3 mm and about 1.5 mm, between about 0.4 mm and about 1 mm. In one embodiment, height h₁ of first sidewall segment 15 is greater than height h₂ of second sidewall segment 25.

Indeed, second sidewall segment 25 bounds a second volume v₂ for holding a liquid and/or a polymerizable liquid. More specifically, second sidewall segment 25 and a plane of first ledge 30 bound a second volume v₂, or said another way the space above first volume v₁ that is bounded by second sidewall segment 25 constitutes second volume v₂. In one embodiment, first volume v₁ is equal to second volume v₂. In one embodiment, first volume v₁ is within +10% of second volume V₂.

First sidewall segment 15 may extend at a right angle upward from bottom wall 9 toward or in the direction of opening 7. Preferably, first sidewall segment 15 forms an obtuse angle with bottom wall 9 (e.g. 91°, 92°, 93°, 94°, 95°, 96°, 97°, 98°, 99°, or more; or less than 99°, 97°, 95°, or 93°). Second sidewall segment 25 may extend at a right angle upward from first ledge 30 (or relative to a plane of bottom wall 9) toward or in the direction of opening 7. Preferably, second sidewall segment 25 forms an obtuse angle with first ledge 30 (or with the plane defined by bottom wall 9) (e.g. 91°, 92°, 93°, 94°, 95°, 96°, 97°, 98°, 99°, or more; or less than 99°, 97°, 95°, or 93°). In one embodiment, both first sidewall segment 15 forms an obtuse angle with bottom wall 9 and second sidewall segment 25 forms an obtuse angle with first ledge 30 (or with the plane defined by bottom wall 9). In one embodiment, only one of first sidewall segment 15 or second sidewall segment 25 forms an obtuse angle respectively with bottom wall 9 or first ledge 30, and the other extends at a right angle relative to a plane of bottom wall 9.

Cell culture apparatus 1 may further comprise a third sidewall segment 35. In one embodiment, third sidewall segment 35 is situated above both first sidewall segment 15 and immediately above second sidewall segment 25 when cell culture apparatus 1 is viewed normal to the depth axis d of well 5. Nevertheless, third sidewall segment 35 is connected to second sidewall segment 25 by a second ledge 40 that extends outward from second sidewall segment 25, relative to a center point of bottom wall 9. At the point where third sidewall segment 35 connects second ledge 40, third sidewall segment 35 extends from second ledge 40 toward or in the direction of opening 7.

Thus, third sidewall segment 35 is dimensionally wider (taken in the plane of bottom wall 9 and/or opening 7) than first sidewall segment 15 and second sidewall segment 25. Regardless of the cross-sectional shape of the first, second or third sidewall segments (as viewed from the direction of opening 7 toward bottom wall 9), if not otherwise stated a measure of distance between two points on a respective sidewall segment is taken where the measure would be the maximal. Thus, in the case of quadrilateral the measure is taken along the longest edge, and in the case of a circle the measure is taken along the diameter. Accordingly, where a cross-sectional shape of third sidewall segment is a circle, a diameter of third sidewall segment 35 may be greater than a diameter of second sidewall segment 25 (and first sidewall segment 15).

In one embodiment, a diameter of third sidewall segment 35 is between about 5 mm and 40 mm (±1 mm), between about 5.25 mm and 35 mm (±1 mm), between about 5.5 mm and 30 mm (±1 mm), between about 5.75 mm and 30 mm (±1 mm), between about 6 mm and 30 mm (±1 mm), or between about 6 mm and 16 mm (±1 mm).

In embodiments where well 5 is comprised in a 96-well plate format, a diameter of third sidewall segment 35 may be between about 3 mm and about 9 mm (±1 mm), between about 4 mm and about 8 mm (±1 mm), or between about 5 mm and about 7 mm (±1 mm).

In embodiments where well 5 is comprised in a 24-well plate format, a diameter of third sidewall segment 35 may be between about 10 mm and about 20 mm (±1 mm), between about 11 mm and about 19 mm (±1 mm), between about 12 mm and about 18 mm (±1 mm), or between about 13 mm and about 17 mm (±1 mm).

In some embodiments, well 5 is comprised in a plate format other than a 96- or 24-well, such as a 48-well plate, a 12-well plate, a 6-well plate, or a petri-type dish, and in such cases a diameter of third sidewall segment 35 may be as set out in the foregoing or may scale accordingly.

In one embodiment, third sidewall segment is polygonal (as viewed in the axis from opening 7 toward bottom wall 9), such as a quadrilateral, pentagon, hexagon, octagon, and so on. In one embodiment, third sidewall segment is rounded or circular (as viewed in the axis from opening 7 toward bottom wall 9).

Second ledge 40 connects a bottom end/edge or rim 42 (i.e. the edge that is closer to bottom wall 9) of third sidewall segment 35 and an upper end/edge or rim 44 (i.e. the edge furthest from bottom wall 9) of second sidewall segment 25. In one embodiment, a width of second ledge w₂ may be between about 0.2 mm and about 5 mm, between about 0.4 mm and about 4 mm, between about 0.6 mm and about 3 mm, or between about 0.7 mm and about 2.5 mm.

In embodiments where well 5 is comprised in a 96-well plate format, a width of second ledge w₂ may be between about 0.2 mm and about 1.5 mm, between about 0.4 mm and about 1.25 mm, or between about 0.6 mm and about 1 mm.

In embodiments where well 5 is comprised in a 24-well plate format, a width of first ledge w₁ may be between about 0.5 mm and about 5 mm, between about 0.75 mm and about 4 mm, between about 1 mm and about 3 mm, or between about 1.25 mm and about 2 mm.

In some embodiments well 5 is comprised in a plate format other than a 96- or 24-well, such as a 48-well plate, a 12-well plate, a 6-well plate, or a petri-type dish then width of first ledge w₂ may be as set out in the foregoing or may scale accordingly.

A height h₃ of third sidewall segment 35, if present, is only limited by the dimension of cell culture apparatus 1, and more specifically by a depth of well 5. In one embodiment, height h₃ of third sidewall 35 is dependent on the sum of height h₁ of first sidewall segment 15 and height h₂ of second sidewall segment 25. In one embodiment, height h₃ of third sidewall segment 35 is between about 1 mm and about 16 mm, between about 1.5 mm and about 9 mm, between about 2 mm and about 8.5 mm, or between about 2.25 mm and about 7 mm. In one embodiment, height h₃ of third sidewall segment 35 is greater than height h₂ of second sidewall segment 25. In one embodiment, height h₃ of third sidewall portion 35 is greater than height h₁ of first sidewall segment 15. In one embodiment, height h₃ of third sidewall segment 35 is greater than the sum of height h₂ of second sidewall segment 25 and height h₁ of first sidewall segment 15.

As with first sidewall segment 15 and second sidewall segment 25, third sidewall segment 35 bounds a third volume v₃ for holding a liquid and/or a polymerizable liquid. More specifically, third sidewall segment 35 and a plane of second ledge 40 bound a third volume v₃, or said another way the space above second volume v₂ that is bounded by third sidewall segment 35 constitutes third volume v₃. In one embodiment, third volume v₃ is greater than second volume v₂. In one embodiment, third volume v₃ is greater than first volume v₁. In one embodiment, third volume v₃ is greater than the sum of second volume v₂ and first volume v₁.

Third sidewall segment 35 may extend at a right angle relative to a plane of bottom wall 9 and/or second ledge 40, toward or in the direction of opening 7. Preferably, third sidewall segment 35 forms an obtuse angle relative to a plane of bottom wall 9 and/or second ledge 40. In one embodiment, each of first sidewall segment 15, second sidewall segment 25, and third sidewall segment 35 form an obtuse angle relative to a plane of bottom wall 9. In one embodiment, only one or two of first sidewall segment 15, second sidewall segment 25, and third sidewall segment 35 form an obtuse angle relative to a plane of bottom wall 9, and the other(s) extend at a right angle relative to a plane of bottom wall 9.

While the foregoing description focused on the features and architecture of a single well 5 of cell culture apparatus 1, it is within the contemplation of this disclosure that a plurality of such wells may be comprised in cell culture apparatus 1. In one embodiment, cell culture apparatus 1 is dimensioned substantially in accordance with the criteria of the American National Standards Institute (ANSI). In one embodiment, cell culture apparatus 1 is a 96-well plate and each well thereof comprises the features and characteristics of certain of the embodiments descried above. In one embodiment, cell culture apparatus 1 is a 24-well plate and each well thereof comprises the features and characteristics of certain of the embodiment descried above. In one embodiment, cell culture apparatus 1 is a 6-well plate and each well thereof comprises the features and characteristics of certain of the embodiment descried above.

Cell culture apparatus 1 may further comprise a lid. In one embodiment, lid and apparatus 1 are designed to reduce or limit evaporation from wells 5, in particular from edge and corner wells. Evaporation results in osmolality changes in the media contained wit in the wells 5, or detriment changes in nutrient or waste by product concentrations. In one embodiment, evaporation is reduced or limited by improving the fit between lid and a lid-bearing surface of cell culture apparatus 1, such as by minimizing a space therebetween.

As will be further described hereinbelow, cell culture apparatus 1 may be used in various cell culture applications. In one embodiment, cells may be seeded directly on bottom wall 9, and subsequently cell culture medium may be added to well 5, such as to the level of first ledge 30 or second ledge 40, or beyond (in embodiments comprising a third wall portion 35). In one embodiment, at least bottom wall 9 is tissue culture treated. In one embodiment, at least bottom wall 9 is not tissue culture treated. In such embodiments, depending on the type of cell to be cultured bottom, wall 9 may be coated with an extracellular matrix, at least one extracellular matrix protein, or a hydrogel.

In one embodiment, an extracellular matrix (or at least one extracellular matrix protein or a hydrogel) may be added into first volume v₁, and cells may be seeded thereon. In one embodiment, an extracellular matrix (or at least one extracellular matrix protein) and one or more cells may be added into first volume v₁, and cells may be seeded therein. In one embodiment, an extracellular matrix (or at least one extracellular matrix protein) is filled up to first ledge 30 or second ledge 40.

As different extracellular matrices possess different mechanisms of gelation, unique challenges arise depending on the type of extracellular matrix and the type of cell culture vessel or container being used. Indeed, certain extracellular matrix products rely on temperature to polymerize, such as Matrige™, which are liquid at lower temperatures and polymerize at higher temperatures. Thus, depending on the rate of temperature increase the shape and localization of thermo-gelling matrices, such as Matrigel™ dispensed on or in the vessel or container is unpredictable. Moreover, for extracellular matrix that polymerize by photocrosslinking, chemical activation, enzymatic action, or introduction of complementary interacting domains, flat culture surfaces on which they are applied do not maintain a desired shape until such time that polymerization may be induced.

An advantage of cell culture apparatus 1 of this disclosure is that first sidewall segment 15 and/or second sidewall segment 25 confine the geometry of a polymerized extracellular matrix protein. Indeed, extracellular matrix is bounded by a sidewall segment, however, a height of extracellular matrix at any point along its surface area exposed to the air (before culture medium is added) may also be controlled when a pre-determined volume of extracellular matrix fills first volume v₁ (or first volume v₁ and second volume v₂).

A further advantage of cell culture apparatus of this disclosure is that by virtue of controlling the shape and height of a polymerized extracellular matrix, variability due to mass transfer rates is limited or eliminated. In turn, three-dimensional cell assemblies or constructs, such as organoids, formed/derived therein may be more uniform in terms of size, in comparison to current state of the art dome approaches. Moreover, three-dimensional cell assemblies or constructs, such as organoids, formed/derived in cell culture apparatus of this disclosure may also exhibit improved seeding efficiency, growth, and/or function.

A still further advantage of cell culture apparatus of this disclosure is that the constrained localization of the region and volume bounded by first sidewall segment 15 permits reliable imaging, due to the predictability of where a culture of cells will be and reduction or elimination of meniscus effects.

### Methods

In other aspects of this disclosure are provided methods or processes of culturing or assaying cells using cell culture apparatus 1 as described above. In a specific aspect, the methods or processes relate to culturing or assaying cells using cell culture apparatus 1 whether under non-adherent conditions, in three dimension, or embedded in a polymerized extracellular matrix, extracellular matrix protein, or hydrogel. In one embodiment, the cells in culture are multicellular aggregates or organoids, and such aggregates/organoids may develop or form while cultured in cell culture apparatus 1.

In one embodiment, a method of this disclosure comprises seeding a population of cells in a well 5 of cell culture apparatus 1.

The population of cells that may be cultured in accordance with the methods/processes of this disclosure are not particularly limited. In certain embodiments the cells require or benefit from exposure to an extracellular matrix, and extracellular matrix component, or a hydrogel. In one embodiment, the cells cultured in accordance with the methods/processes of this disclosure are mammalian cells, such as human, non-human primate, rodent cells, or otherwise.

In one embodiment, the population of cells cultured in accordance with the methods/processes of this disclosure are mammalian epithelial cells, such as mammalian epithelial stem or progenitor cells. In one embodiment, the mammalian epithelial stem or progenitor cells form organoids as they are cultured in a cell culture apparatus of this disclosure, in accordance with the method disclosed herein. In one embodiment, the mammalian epithelial stem or progenitor cells form organoids while embedded in an extracellular matrix, extracellular matrix component, or hydrogel having been added to a cell culture apparatus of this disclosure.

In one embodiment, the population of cells cultured in accordance with the methods/processes of this disclosure are primary, that is they are obtained from a patient, subject, biopsy, or otherwise. In one embodiment, the cells cultured in accordance with the methods/processes of this disclosure are pluripotent stem cells (PSC), such as induced PSC or embryonic stem cells. In one embodiment, the cells cultured in accordance with the methods/process of this disclosure are PSC-derived, that is to say that the cells at one point were undifferentiated pluripotent stem cells but were subsequently differentiated to a downstream lineage of interest (e.g. by way of non-limiting example, an epithelial-like stem cell).

Methods of this disclosure may further comprise adding a liquid extracellular matrix, one or more liquid extracellular matrix proteins, or a liquid hydrogel to first volume v₁ bounded by first sidewall segment 15. In a specific embodiment, first volume v₁ is filled with the liquid extracellular matrix, one or more liquid extracellular matrix proteins, or the liquid hydrogel. In such an embodiment, the liquid extracellular matrix, one or more liquid extracellular matrix proteins, or the liquid hydrogel may be added up to a level of first ledge 30.

In one embodiment, a liquid extracellular matrix, one or more liquid extracellular matrix proteins, or a liquid hydrogel may be added or filled beyond first volume v₁, such as into second volume v₂. In one embodiment, second volume v₂ is filled with a liquid extracellular matrix, one or more liquid extracellular matrix proteins, or a liquid hydrogel. In one embodiment, first volume v₁ and second volume v₂ are filled sequentially with the same or different liquid extracellular matrix, one or more liquid extracellular matrix proteins, or liquid hydrogel.

In one embodiment, the population of cells are suspended in the extracellular matrix, one or more extracellular matrix proteins, or the hydrogel. In one embodiment, the population of cells are suspended in the extracellular matrix, one or more extracellular matrix proteins, or the hydrogel before it is added to or fills first volume v₁ (or first volume v₁ and second volume v₂). In one embodiment, the population of cells are embedded in the extracellular matrix, one or more extracellular matrix proteins, or the hydrogel. In one embodiment, the population of cells are sandwiched between a first layer of extracellular matrix, one or more extracellular matrix proteins, or the hydrogel in volume v₁ and a second layer of extracellular matrix, one or more extracellular matrix proteins, or the hydrogel in volume v₂.

Methods of this disclosure may further comprise polymerizing the extracellular matrix protein, one or more liquid extracellular matrix proteins or the liquid hydrogel (after it is added to or fills first volume v₁, or first volume v₁ and second volume v₂). Polymerizing the extracellular matrix protein, one or more liquid extracellular matrix proteins or the liquid hydrogel (after it is added to or fills first volume v₁, or first volume v₁ and second volume v₂) may comprise incubating, such as at a temperature amenable to polymerization of the extracellular matrix protein, one or more liquid extracellular matrix proteins or the liquid hydrogel.

Once the liquid extracellular matrix protein, one or more liquid extracellular matrix proteins or the liquid hydrogel is added to or fills first volume v₁, or first volume v₁ and second volume v₂, and optionally has polymerized, the population of cells may be exposed to a cell culture medium. In one embodiment, the population of cells are seeded on a polymerized extracellular matrix, one or more extracellular matrix proteins or the hydrogel, and may thus be directly exposed to a cell culture medium. In one embodiment, the population of cells are seeded or embedded in a polymerized extracellular matrix, one or more extracellular matrix proteins or the hydrogel, and may thus be indirectly exposed to a cell culture medium, such as by diffusion.

In one embodiment, cell culture apparatus 1 comprises a polymerized extracellular matrix, one or more extracellular matrix proteins or a hydrogel (and a population of cells thereon or therein) up to a level of first ledge 30, and a cell culture medium is added to second volume v₂ bounded by second sidewall segment 25, and possibly into third volume v₃ bounded by third sidewall segment 35.

In one embodiment, cell culture apparatus 1 comprises a polymerized extracellular matrix, one or more extracellular matrix proteins or a hydrogel (and a population of cells thereon or therein) up to a level of first ledge 30, and second volume v₂ bounded by second sidewall segment 25 is filled with the cell culture medium. In such an embodiment, a volume of the extracellular matrix, one or more extracellular matrix proteins, or hydrogel (in fist volume v₁) is equal to a volume of the cell culture medium (in second volume v₂).

Thus, after a sufficient exposure time to the culture environment (e.g. extracellular matrix and cell culture medium), the population of cells may develop or form into one or more multicellular aggregates or organoids. A sufficient exposure time is typically measured in days or weeks, such as 3 days or 5 days, or 1 week, 2 weeks, 3 weeks, or more.

Cell culture media are widely known and available, including organoid media, as have been commercialized by STEMCELL Technologies including those media products commercialized under the IntestiCult™, HepatiCult™, PneumaCult™, PancreaCult™ and STEMdiff™ brands. some embodiments, not a cell culture medium but a different cell culture reagent may be added (or filled) into second volume v₁, or second volume v₂ and third volume v₃. Non-limiting examples may include digestive enzymes, or other reagents that may be used in downstream analysis of culture of cells, such as those that may be required for staining operations, viability assessments, etc.

In one embodiment, the population of cells may be treated with one or more test compounds and one or more control compounds (as may be diluted in an appropriate culture medium), to assess toxicity or responsiveness to the candidate compound(s). In one embodiment, one or more test compounds and one or more control compounds are respectively comprised in a cell culture medium to which the population of cells are (directly or indirectly) exposed in respective wells of a cell culture apparatus of this disclosure.

Accordingly, cells cultured in accordance with the foregoing methods using cell culture apparatus 1 as described herein may possess several advantages over conventional approaches, particularly when forming or assaying multicellular aggregates or organoids. Indeed, by virtue of controlling the shape and height of a polymerized extracellular matrix (or ecm protein, or hydrogel), culture performance variability due to mass transfer rates may be limited or eliminated. In turn, three-dimensional cell assemblies or constructs, such as organoids, formed/derived therein may be more uniform in terms of size and/or function, in comparison to current state of the art dome approaches. Moreover, three-dimensional cell assemblies or constructs, such as organoids, formed/derived in cell culture apparatus of this disclosure may also exhibit improved seeding efficiency, growth, and/or function.

In addition, in the case of matrices, matrix protein(s), or hydrogels that rely on temperature for gelation, cell culture apparatus of this disclosure may reduce or eliminate the need to be pre-warmed because shape and position of the matrices are constrained by the geometry of the apparatus while in their unpolymerized state. Ordinarily, plates are pre-warmed so that the matrix more rapidly polymerizes in its original placement, thereby attempting to decrease the opportunity for domes or droplets to spread, shift, or deform prior to polymerization in ways that can increase the variability of the culture.

In addition, practice of the methods (using cell culture apparatus 1) may facilitate automation, such as imaging and analysis operations, due to the increased control over matrix and cell deposition/localization.

In one embodiment, automated methods of preparing and seeding a plurality of wells (of a cell culture apparatus as described herein) may rely on automated liquid handlers, as are known in the art.

In one embodiment, automated methods of assaying a culture of cells in a plurality of wells (of a cell culture apparatus as described herein) may rely on automated imagers affixed to a gantry or the like. Conversely, automated methods of assaying a culture of cells in a plurality of wells (of a cell culture apparatus as described herein) may rely on a stationary imager and a movable stage on which to rest a cell culture apparatus of this disclosure.

The following non-limiting examples are illustrative of the present disclosure.

### EXAMPLES

### Example 1: Optimizing Extracellular Matrix Depth (Height of the First Sidewall Portion)

Cell culture apparatus of this disclosure were manufactured by injection molding or by 3D printing. In the case of 3D printing, a base and a well-defining member were formed using an Ultimaker S5 printer with Tough PLA filament, and the two components were subsequently attached to one another. In some cases, such as when 3D printed, cell culture apparatus of this disclosure were sterilized by UV exposure before use.

In this example, the effects of varying a height of the first sidewall portion was explored, and wells were manufactured having first sidewall portion heights of 0.75 mm, 1 mm, 1.25 mm, and 1.5 mm.

Human hepatic organoids and human colon organoids were generated in accordance with the manufacturer's recommendations in HepatiCult™ OGM (STEMCELL Technologies) or IntestiCult™ OGM (STEMCELL Technologies), respectively. On the day of the experiment the organoids were dissociated into small clumps, as directed by the manufacturer, and suspended in either 100% or 50% growth factor reduced Matrigel®. The cell suspension was added to each cell culture apparatus design up to the first ledge or the second ledge, or applied as a dome on a bottom wall of a standard 24-well plate. After the extracellular matrix polymerized, 500 μL of either HepatiCult™ OGM (STEMCELL Technologies) or IntestiCult™ OGM (STEMCELL Technologies) was added to each well.

Each well was imaged on days 3, 5 and 7 using tiling on a widefield Zeiss microscope at 5× and stitched into a whole well image. Whole well images were analyzed using a Fiji macros to output the average cross-sectional area of all organoids within a culture well.

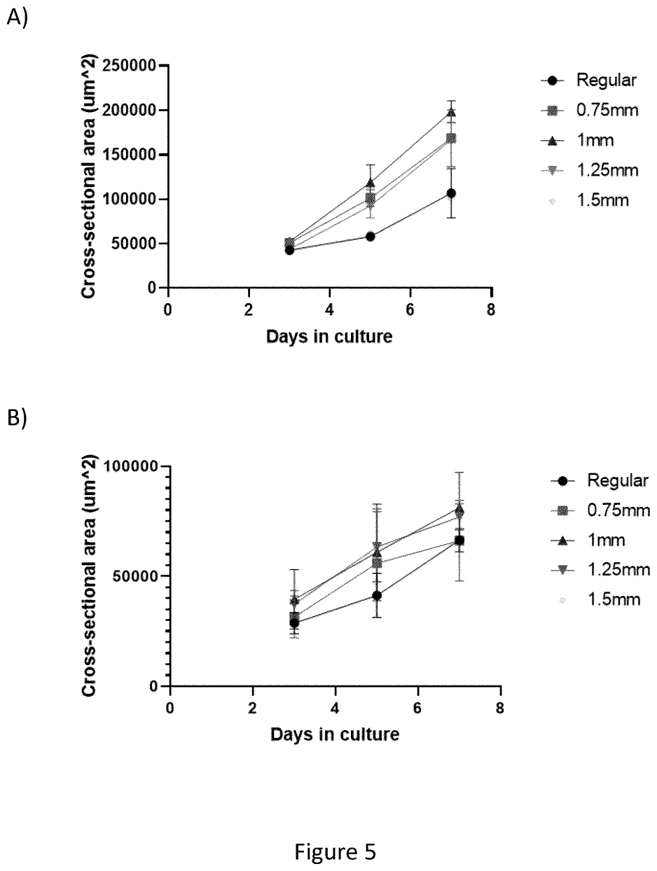

Hepatic and colon organoid growth in cell culture apparatus of this disclosure generally outperformed the standard dome culture method (“Regular”), with the 0.75 mm, 1 mm and 1.25 mm embodiments outperforming the 1.5 mm embodiment (FIG. 5A and B).

Example 2: Characterizing growth of organoids in cell culture apparatus of this disclosure Human intestinal organoids were maintained in IntestiCult™ OGM (STEMCELL Technologies) and dissociated as recommended by the manufacturer. An arising cell suspension of clumps was mixed with 50% growth factor reduced Matrigel solution and 50 μl was either filled in the volume bounded by first sidewall portion of a 24-well format cell culture apparatus of this disclosure or deposited as a dome on a conventional flat bottom 24-well plate. After the extracellular matrix polymerized, 500 μL of IntestiCult™ OGM (STEMCELL Technologies) was added to each well.

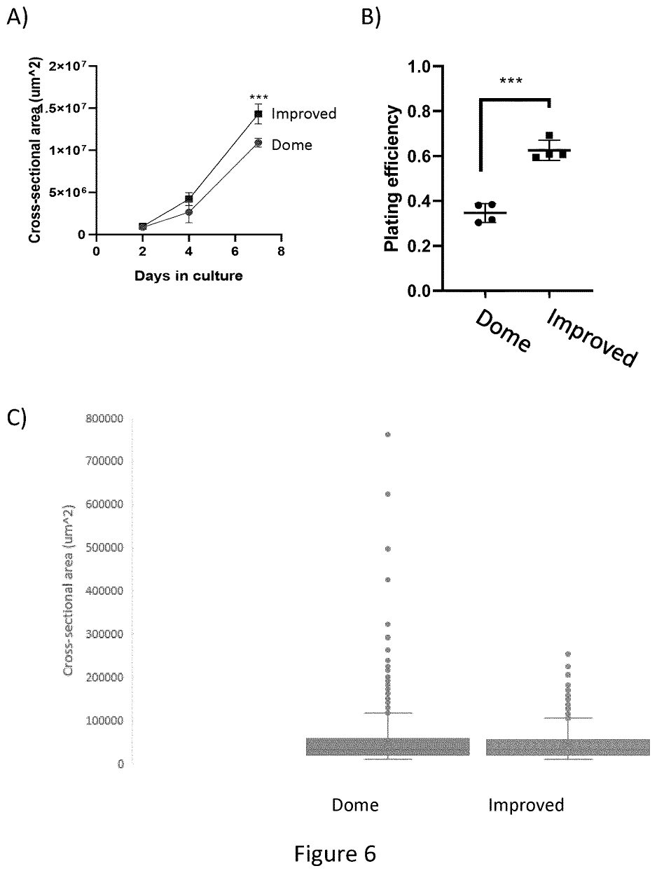

To assess growth, each well as imaged and analyzed as in Example 1. Cell culture apparatus of this disclosure show statistically greater growth by cross-sectional area compared to conventional dome culture (FIG. 6A).

To assess seeding efficiency, day 7 organoids were imaged and analyzed as in Example 1 and the number of clumps seeded at DO was divided by the counted number of organoids greater than 100 μm in diameter in the day 7 culture. Cell culture apparatus of this disclosure show statistically greater plating efficiency compared to conventional dome culture (FIG. 6B).

To assess organoid size distribution, day 7 organoids were imaged and analyzed as in Examples 1 and 2. Cell culture apparatus of this disclosure routinely show reduced variation in size distribution as measured by average cross-sectional area per culture (FIG. 6C). In contrast to dome cultured organoids, organoids cultured in cell culture apparatus of this disclosure do not appear to grow/develop into extremely large organoids.

### Example 3: Assessing Functionality of Organoids in Cell Culture Apparatus of This Disclosure

Human intestinal organoids were dissociated and seeded essentially as described in Example 2. Responsiveness of the organoids to forskolin (Orkambi™ and Gefitinib, data not shown) was assessed. Day 7 organoids in either a 24-well plate format of a cell culture apparatus of this disclosure or deposited as a dome in a standard 24-well plate were exposed to either 5 μM forskolin or DMSO for approximately 90 minutes. At various time points each well was imaged and analyzed as described in Examples 1 and 2 to determine an average cross sectional area of the organoids in culture.

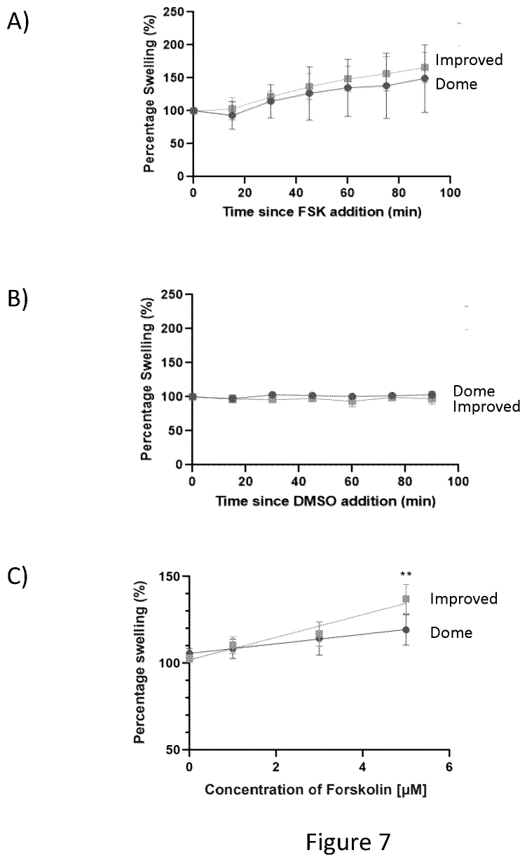

Human intestinal organoids exposed to forskolin exhibited slightly improved swelling in a cell culture apparatus of this disclosure in comparison to standard dome culture. Notably the standard deviation was greatly reduced among organoids treated in a cell culture apparatus of this disclosure in comparison to standard dome culture (FIG. 7A and B). A dose-response experiment was performed for human intestinal organoids exposed to different doses of forskolin in either a 96-well plate format of a cell culture apparatus of this disclosure or a standard dome culture. Organoids assed in a cell culture apparatus of this disclosure showed a significantly more robust response to forskolin than organoids assessed in standard dome culture (FIG. 7C).

### Example 4: Extracellular Matrix Retention in Cell Culture Apparatus of This Disclosure

Human hepatic organoids were dissociated essentially as described in Example 1. An arising cell suspension of clumps was mixed with either 100%, 75%, 50%, 20%, and 10% growth factor reduced Matrigel solution, and 50 μl was either filled in the volume bounded by first sidewall portion of a 24-well format cell culture apparatus of this disclosure or deposited as a dome on a conventional flat bottom 24-well plate. After the extracellular matrix polymerized, 500 μL of HepatiCult™ OGM (STEMCELL Technologies) was added to each well, with full medium changes every 2-3 days. Cultures were imaged on day 4 and day 7 using tiling on a widefield Zeiss microscope at 5× and stitched into a whole well image.

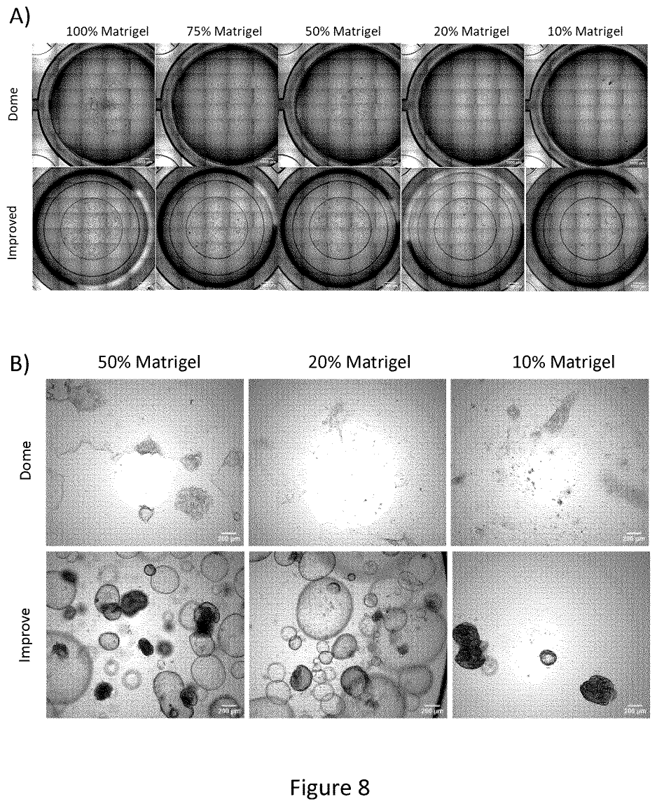

Cell culture apparatus of this disclosure (“Improved”) are able to support the deposition and gelation of lower concentrations/higher dilutions of Matrigel than can be successfully formed using dome culture methods (FIG. 8A and B). Furthermore, at high Matrigel dilution cell culture apparatus of this disclosure support greater organoid growth than traditional dome culture, and suffer fewer (often no) disruptions or dislodgings of the gelled matrix volume than dome culture methods (FIG. 8A and B).

Example 5: Quantifying Evaporation Levels From Cell Culture Apparatus of this Disclosure Either standard 24- and 96-well culture plates, or 24- and 96 -well cell culture apparatus of this disclosure designed with a better fitting lid that reduced spacing between an underside of the lid and the lid-bearing surface of the plate were filled with 500 μL and 200 μL of distilled water coloured with red dye, respectively. Daily absorbance measurements were taken at 504 nm over 7 days, and the concentration of red dye was correlated with absorbance according to the Beer-Lambert Law. “Corner” wells corresponded to each of the four corners. “Edge” wells corresponded to the wells of the outer perimeter that are not at the corners. “Inner” wells represented all remaining wells.

FIG. 8 shows that in each plate format (24- or 96 -well) cell culture apparatus of this disclosure demonstrated significantly less evaporation rates among corner and edge wells in comparison to the corresponding standard culture plate.

The above-described example embodiments of the present disclosure are intended to be illustrative only and in no way limiting. The described embodiments are susceptible to many modifications of composition, details, and order of operation. The invention, rather, is intended to encompass all such modifications within its scope, as defined by the claims, which should be given a broad interpretation consistent with the description as a whole.

1. A cell culture apparatus, comprising
a) a well having a bottom wall and an opening:
b) a first sidewall segment connected to the bottom wall and extending in a direction away from the bottom wall, the first sidewall segment and the bottom wall bounding a first volume for holding a liquid and/or a polymerizable liquid; and
c) a second sidewall segment connected to the first sidewall segment by a first ledge, the second sidewall segment and a plane of the first ledge bounding a second volume for holding a liquid and/or a polymerizable liquid,
wherein a height of the first sidewall segment is greater than a height of the second sidewall segment.

2. The cell culture apparatus of claim 1, wherein the first volume is equal ±10% to the second volume.

3. The cell culture apparatus of claim 1, wherein
a) the height of the first sidewall segment is between about 0.1 mm and 10 mm, preferably between 0.5 mm and 5 mm; and/or
b) the height of the second sidewall segment is between about 0.1 mm and 10 mm, preferably between 0.2 mm and 4 mm.

4. (canceled)

5. The cell culture apparatus of claim 1, wherein the first sidewall segment forms an obtuse angle with the bottom wall, and/or the second sidewall segment forms an obtuse angle with the first ledge.

6. The cell culture apparatus of claim 1, wherein a width of the first ledge is between about 0.1 mm to about 5 mm.

7. The cell culture apparatus of claim 1, wherein one or both of the first sidewall segment and the second sidewall segment are circular or substantially circular in the plane of the bottom wall, and wherein a diameter of the first sidewall segment at any position along the height thereof is smaller than a diameter of the second sidewall segment at any position along the height thereof.

8. (canceled)

9. (canceled)

10. (canceled)

11. The cell culture apparatus of any one of claim 1, further comprising a third sidewall segment connected to the second sidewall segment by a second ledge, the third sidewall segment and a plane of the second ledge bounding a third volume for holding a liquid and/or a polymerizable liquid.

12. The cell culture apparatus of claim 11, wherein the third sidewall segment extends from the second ledge to the opening.

13. The cell culture apparatus of claim 11, wherein a height of the third sidewall segment is greater than the height of the second sidewall segment, and the third volume is greater than the second volume.

14. The cell culture apparatus of claims 11, wherein the height of the third sidewall segment is between about 1 to 15 mm, or between about 2 to 10 mm.

15. The cell culture apparatus of claim 11, wherein the second ledge and the third sidewall segment form an obtuse angle.

16. The cell culture apparatus of claim 11, wherein a width of the second ledge is between about 0.2 mm and about 5 mm.

17. The cell culture apparatus of claim 11, wherein the third sidewall segment is substantially circular in the plane of the bottom wall, and wherein a diameter of the third sidewall segment at any position along the height thereof is larger than the diameter of the second sidewall segment at any position along the height thereof.

18. The cell culture apparatus of claim 1, further comprising more than one well, each well comprising respectively at least:
a) a bottom wall and an opening:
b) a first sidewall segment connected to the bottom wall and extending in a direction away from the bottom wall, the first sidewall segment and the bottom wall bounding a first volume for holding a liquid and/or a polymerizable liquid; and
c) a second sidewall segment connected to the first sidewall segment by a first ledge, the second sidewall segment and a plane of the first ledge bounding a second volume for holding a liquid and/or a polymerizable liquid,
wherein a height of the first sidewall segment is greater than a height of the second sidewall segment.

19. A method of culturing cells comprising seeding a population of cells in a well of a cell culture apparatus as described in of claim 1.

20. The method of claim 19, further comprising:
adding a liquid extracellular matrix, one or more liquid extracellular matrix proteins, or a liquid hydrogel to the first volume bounded by the first sidewall segment of the cell culture apparatus; and
polymerizing the liquid extracellular matrix, the one or more liquid extracellular matrix proteins, or the liquid hydrogel.

21. (canceled)

22. The method of claim 1, wherein the population of cells are suspended and/or embedded in the extracellular matrix, the one or more extracellular matrix proteins, or the hydrogel.

23. The method of claim 22, further comprising exposing the population of cells to a cell culture medium by adding the cell culture medium to the second volume bounded by the second sidewall segment.

24. The method of claim 23, wherein a volume of the extracellular matrix, the one or more extracellular matrix proteins, or the hydrogel is equal to a volume of the cell culture medium.

25. The method of claim 23, further comprising generating a multicellular aggregate or an organoid from the population of cells.
